(19) United States
(12) Patent Application Publication
(10) Pub. No.: US 2014/0031917 A1
(43) Pub. Date: Jan. 30, 2014

(54) Matched End Stiffness Stent and Method of Manufacture

(71) Applicant: Dustin Thompson, Santa Rosa, CA (US)

(72) Inventor: Dustin Thompson, Santa Rosa, CA (US)

(73) Assignee: Medtronic Vascular, Inc., Santa Rosa, CA (US)

(21) Appl. No.: 13/557,823

(22) Filed: Jul. 25, 2012

(51) Int. Cl. A61F 2/84; A61F 2/82

(52) U.S. Cl. 623/1.11; 623/1.15

(57) ABSTRACT

The matched end stiffness stent system and method of manufacture includes a stent delivery system including a catheter, balloon, and stent. The stent includes a wire bent into a waveform having a constant frequency and wrapped into a hollow cylindrical shape, the wire having a body portion having body struts connected between body crowns, the body struts having substantially equal lengths, and the waveform in the body portion having a constant amplitude; and at least one end portion attached to the body portion, the at least one end portion having end struts connected between end crowns, the waveform in the at least one end portion having an amplitude different from the constant amplitude of the waveform in the body portion. The cross sections of the end struts are selected so that the body struts and the end struts have a substantially equal stiffnesses in response to an applied load.

### TECHNICAL FIELD

The technical field of this disclosure is medical implant devices, particularly, matched end stiffness stent systems and methods of manufacture.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau. Another exemplary wire stent is the Welded Sinusoidal Wave Stent disclosed in U.S. Pat. No. 6,136,023 to Boyle. Balloon-expandable stents are mounted on a collapsed balloon at a diameter smaller than when the stents are deployed. Stents can also be self-expanding, growing to a final diameter when deployed without mechanical assistance from a balloon or like device.

Concern over the long-term effects of stents in the body has led to experimentation with bare metal stents, i.e., stents with no polymers on their exposed surfaces. One fabrication method has been to form stents from a single wire by bending the single wire into a desired shape, such as a sinusoid, wrapping the bent wire around a manifold, then welding adjacent portions of the wire together to form the final stent configuration of a right circular cylinder. Forming a sinusoidal wire into a right circular cylinder results in struts of various lengths. Unfortunately, stents formed from a single wire have different lengths depending upon the portion of the stent in which the wire is used, resulting in different stiffnesses. For example, the ends of the stents have various strut lengths to form a right circular cylinder, but have uniform cross sections. This results in different stiffnesses and flexibility depending upon the length of the strut. Shorter struts are stiffer, while longer struts are less stiff and more susceptible to bending or opening during stent deployment. Due to variations in stiffness, some stent portions expand less than other stent portions during stent deployment and some stent portions react to external loads more than other stent portions react.

It would be desirable to have a matched end stiffness stent system and method of manufacture that would overcome the above disadvantages.

### SUMMARY OF THE INVENTION

One aspect of the present invention provides a stent delivery system reacting to an applied load, the stent delivery system including a catheter; a balloon operably attached to the catheter; and a stent disposed on the balloon. The stent includes a wire bent into a waveform having a constant frequency and wrapped into a hollow cylindrical shape to form the stent, the wire having a body portion having body struts connected between body crowns, the body struts having substantially equal lengths, and the waveform in the body portion having a constant amplitude; and at least one end portion attached to the body portion, the at least one end portion having end struts connected between end crowns, the waveform in the at least one end portion having an amplitude different from the constant amplitude of the waveform in the body portion. The cross sections of the end struts are selected so that the body struts and the end struts have substantially equal stiffnesses in response to the applied load.

Another aspect of the present invention provides a stent including a wire bent into a waveform having a constant frequency and wrapped into a hollow cylindrical shape to form the stent, the wire having a body portion having body struts connected between body crowns, the body struts having substantially equal lengths, and the waveform in the body portion having a constant amplitude; and at least one end portion attached to the body portion, the at least one end portion having end struts connected between end crowns, the waveform and the at least one end portion having an amplitude different from the constant amplitude of the waveform in the body portion. The cross sections of the end struts are selected so that the body struts and the end struts have substantially equal stiffnesses in response to the applied load.

Another aspect of the present invention provides a method of manufacturing a stent from a wire, the stent having a body portion and an end portion, the method including bending the wire into an unwrapped configuration; swaging the wire in selected strut portions in the end portion of the wire, the degree of swaging being selected so that each end strut in the end portion of the stent has a stiffness in response to an applied load substantially equal to a stiffness in response to the applied load of body struts in the body portion of the stent; wrapping the swaged wire about a mandrel to form a hollow cylindrical shape; and selectively welding adjacent segments of the hollow cylindrical shape together to form the stent.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of a stent delivery system made in accordance with the present invention.

FIGS. 2A-2C are a side view of a stent in a wrapped configuration, a side view of a stent in an unwrapped configuration, and a detail of a stent in an unwrapped configuration, respectively, the stent having matched end stiffness in accordance with the present invention.

FIGS. 3A & 3B are a side view of a stent in a wrapped configuration and a side view of a stent in an unwrapped configuration, respectively, the stent having matched end stiffness in accordance with the present invention.

FIGS. 4A-4C are schematic views of loads applied to a stent having matched end stiffness in accordance with the present invention.

FIGS. 5A & 5B are schematic views of models of strut loading for a stent having matched end stiffness in accordance with the present invention.

FIGS. 6A-6D are detail cross section views of wire for a stent with matched end stiffness in accordance with the present invention.

FIGS. 7A-7C are detail views of swaged wire for a stent with matched end stiffness in accordance with the present invention.

FIG. 8 is a flowchart of a method of manufacturing a stent with matched end stiffness in accordance with the present invention.

FIGS. 9A-9E are schematic views of manufacture of a stent with matched end stiffness in accordance with the present invention.

### DETAILED DESCRIPTION OF PRESENTLY PREFERRED EMBODIMENTS

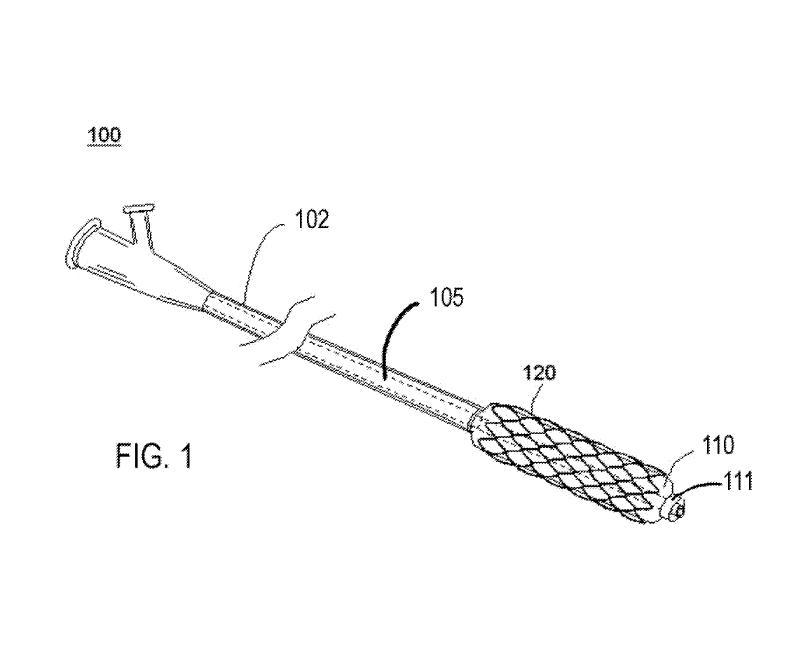

FIG. 1 is a perspective view of a stent delivery system made in accordance with the present invention. The stent delivery system 100 includes a catheter 105, a balloon 110 operably attached to the catheter 105, and a stent 120 disposed on the balloon 110. The balloon 110, shown in an inflated state, can be any variety of balloons capable of expanding the stent 120. The balloon 110 can be manufactured from a material such as polyethylene, polyethylene terephthalate (PET), nylon, Pebax polyether-block co-polyamide polymers, or the like. In one embodiment, the stent delivery system 100 can include retention means 111, such as mechanical or adhesive structures, for retaining the stent 120 on the balloon 110 until the stent 120 is deployed. The catheter 105 may be any variety of balloon catheters, such as a PTCA (percutaneous transluminal coronary angioplasty) balloon catheter, capable of supporting a balloon during angioplasty. The stent delivery system 100 can also include a sheath 102 through which the stent 120 is delivered to the deployment site.

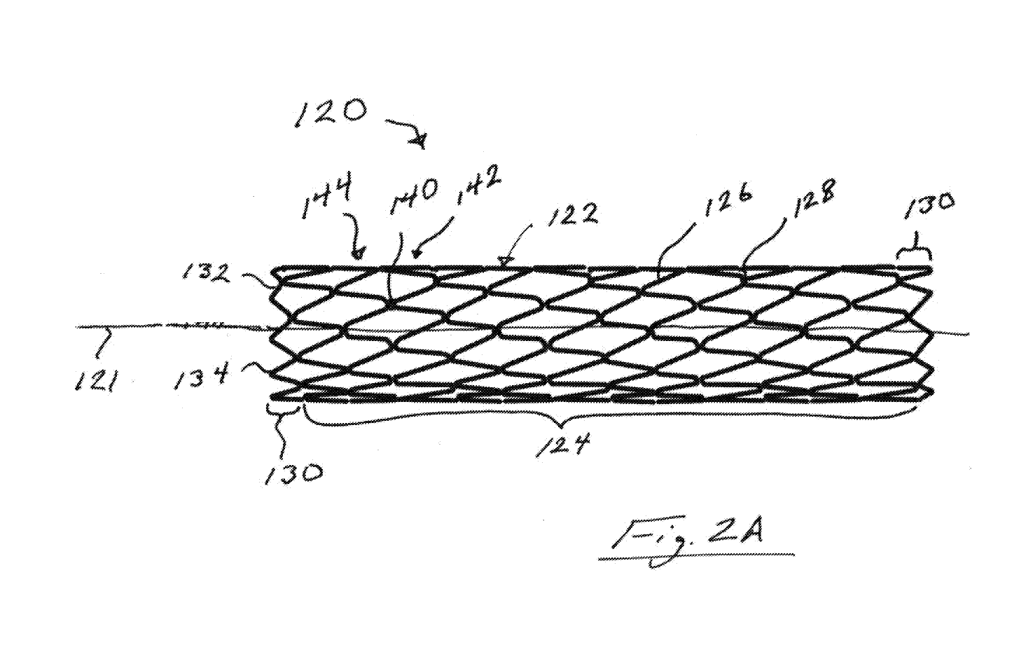

FIGS. 2A-2C, in which like elements share like reference numbers, are a side view of a stent in a wrapped configuration, a side view of a stent in an unwrapped configuration, and a detail of a stent in an unwrapped configuration, respectively, the stent having matched end stiffness in accordance with the present invention. In this example, the struts in the end portion are shorter than the struts in the body portion, and the amplitude of the waveform in the end portion of the stent decreases.

The stent 120 is a wire 122 bent into a waveform having a constant frequency and wrapped into a hollow cylindrical shape to form the stent 120. The wire 122 includes a body portion 124 and end portions 130 attached to the body portion 124. The body portion 124 has body struts 126 connected between body crowns 128. In this example, the body struts 126 have substantially equal lengths. The end portions 130 have end struts 132 connected between end crowns 134. The end struts 132 have stiffnesses substantially equal to the stiffnesses of the body struts 126. The waveform has a constant amplitude in the body portion 124 and amplitude different from that of the body portion 124 in the end portions 130. The body crowns 128 can be welded to body crowns in an adjoining segment of the hollow cylindrical shape of the stent 120, and can be welded to the end crowns 134. The number of welds can be selected to provide the desired longitudinal flexibility to the stent 120, i.e., all the adjacent crowns from one segment to the next need not be welded together. FIG. 2A illustrates an exemplary weld 140 between segment 142 and adjacent segment 144 of the body portion 124.

The end struts 132 have stiffnesses substantially equal to the stiffnesses of the body struts 126 because the cross sections of the end struts 132 and resulting area moment of inertia are selected so that the stiffnesses in response to an applied load are substantially equal. The end struts 132 can be swaged to achieve the desired cross section. As defined herein, “stiffness” is a given load (P) applied to the strut divided by the amount of deflection (w). As further defined herein, parameters are “substantially equal” when the parameters are within plus or minus five percent.

Referring to FIG. 2B, the stent 120 is illustrated in the unwrapped configuration. The length and amplitude of the body portion 124 can be selected to provide the desired longitudinal length of the stent 120 along the axis 121 when in the wrapped configuration. The end portions 130 have decreasing amplitude to square off the ends of the stent 120. In this example, the waveform of the wire 122 is sinusoidal. The body crowns 128 and the end crowns 134 alternate between peaks and valleys moving across the length of the wire 122. Those skilled in the art will appreciate that the waveform can be any periodic function with struts and crowns, and need not be symmetrical about the crown. For example, the waveform can be triangular with the periodic function being a longer strut, a crown, a shorter strut, a crown, and repeating with a longer strut.

The wire 122 of the stent 120 can be made from any biocompatible material used to form a stent such as stainless steel, nickel-cobalt-chromium-molybdenum superalloy, titanium-nickel (nitinol), magnesium, steel alloys containing chromium, cobalt, tungsten, and/or iridium, titanium, cobalt-chromium-platinum, nickel-platinum, molybdenum-rhenium, tantalum, combinations of these materials, or any other biologically compatible low shape-memory material and/or can include composite layers of any of the materials listed.

Referring to FIG. 2C, the amplitude of the end portion 130 in this example decreases linearly. The angle θ between a line 150 passing through the peak crowns of the end portion 130 and the long axis 152 of the wire 122 forms an angle θ which is the same angle at which the wire 122 is wrapped about a mandrel in fabricating the stent 120. This squares off the ends of the stent 120 in the wrapped configuration. Those skilled in the art will appreciate that the angle θ can be selected as desired for a particular application.

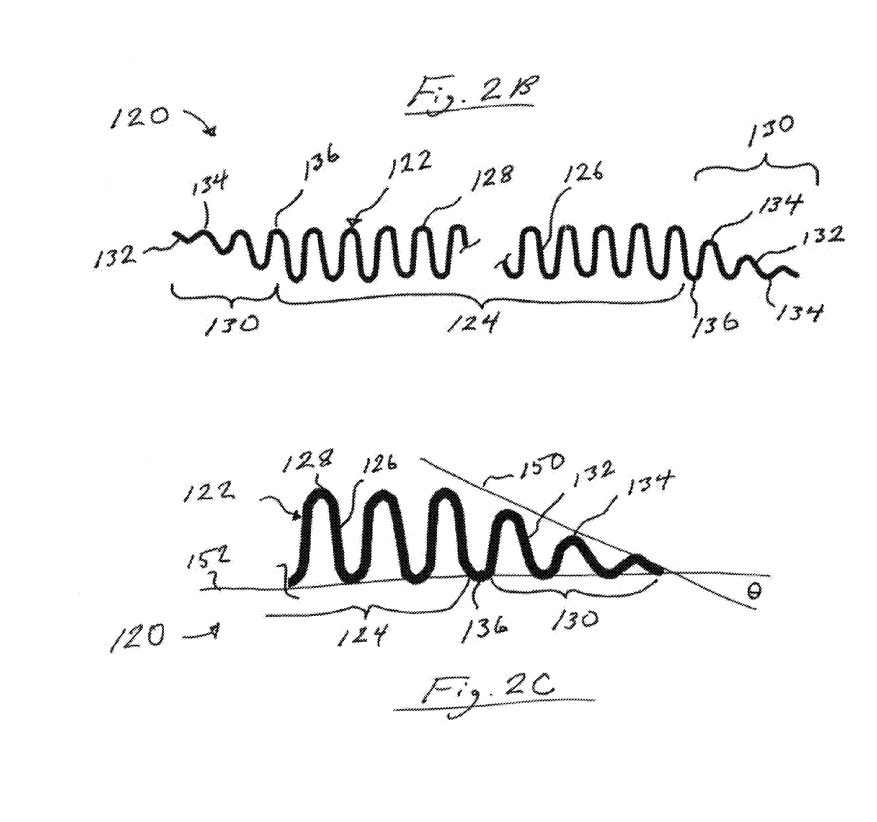

FIGS. 3A & 3B, in which like elements share like reference numbers with each other and with FIGS. 2A-2C, are a side view of a stent in a wrapped configuration and a side view of a stent in an unwrapped configuration, respectively, the stent having matched end stiffness in accordance with the present invention. In this example, the struts in the end portion are longer than the struts in the body portion, and the amplitude of the waveform in the end portion of the stent increases.

The stent 1120 is a wire 122 bent into a waveform having a constant frequency and wrapped into a hollow cylindrical shape to form the stent 1120. The wire 122 includes a body portion 124 and end portions 1130 attached to the body portion 124. The body portion 124 has body struts 126 connected between body crowns 128. In this example, the body struts 126 have substantially equal lengths. The end portions 1130 have end struts 1132 connected between end crowns 1134. The end struts 1132 have stiffnesses substantially equal to the stiffnesses of the body struts 126. The waveform has a constant amplitude in the body portion 124 and amplitude different from that of the body portion 124 in the end portions 1130. The body crowns 128 can be welded to body crowns in an adjoining segment of the hollow cylindrical shape of the stent 1120, and can be welded to the end crowns 1134. The number of welds can be selected to provide the desired longitudinal flexibility to the stent 1120, i.e., all the adjacent crowns from one segment to the next need not be welded together. FIG. 3A illustrates an exemplary weld 140 between segment 142 and adjacent segment 144 of the body portion 124.

The end struts 1132 have stiffnesses substantially equal to the stiffnesses of the body struts 126 because the cross section of the end struts 1132 and resulting area moment of inertia are selected so that the stiffnesses in response to an applied load are substantially equal. The end struts 1132 can be swaged to achieve the desired cross section. As defined herein, “stiffness” is a given load (P) applied to the strut divided by the amount of deflection (w). As further defined herein, parameters are “substantially equal” when the parameters are within plus or minus five percent.

Referring to FIG. 3B, the stent 1120 is illustrated in the unwrapped configuration. The length and amplitude of the body portion 124 can be selected to provide the desired longitudinal length of the stent 1120 along the axis 1121 when in the wrapped configuration. The end portions 1130 have increasing amplitude to square off the ends of the stent 1120. In this example, the waveform of the wire 122 is sinusoidal. The body crowns 128 and the end crowns 1134 alternate between peaks and valleys moving across the length of the wire 122. Those skilled in the art will appreciate that the waveform can be any periodic function with struts and crowns, and need not be symmetrical about the crown. For example, the waveform can be triangular with the periodic function being a longer strut, a crown, a shorter strut, a crown, and repeating with a longer strut.

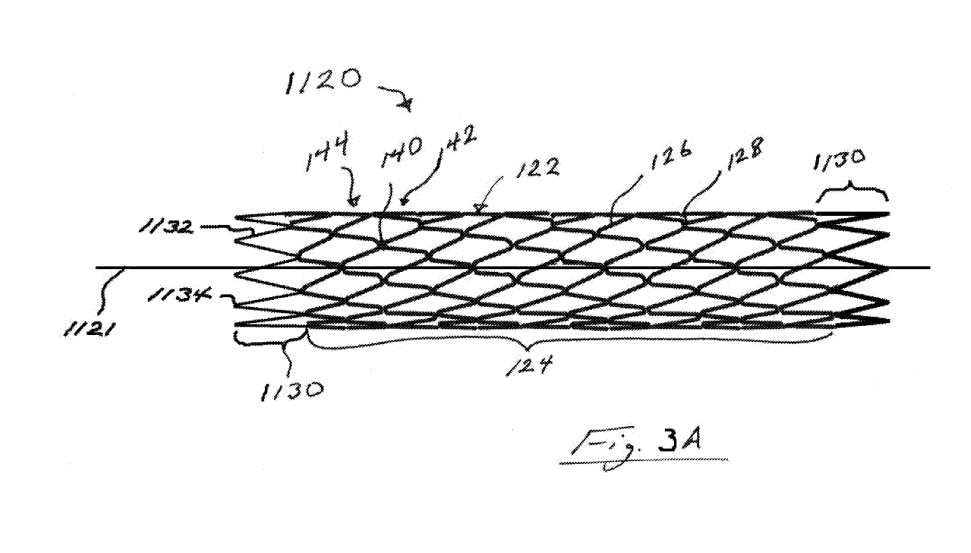

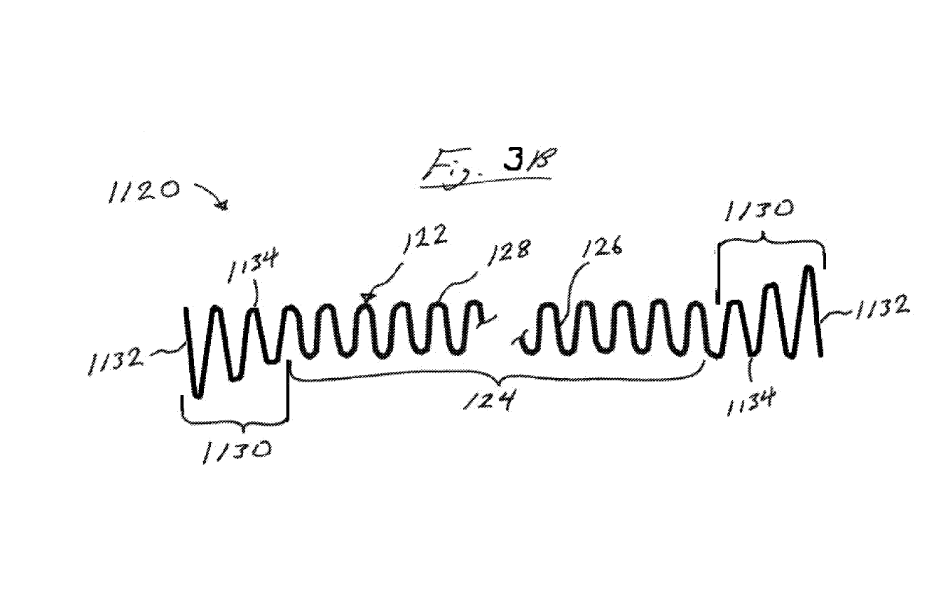

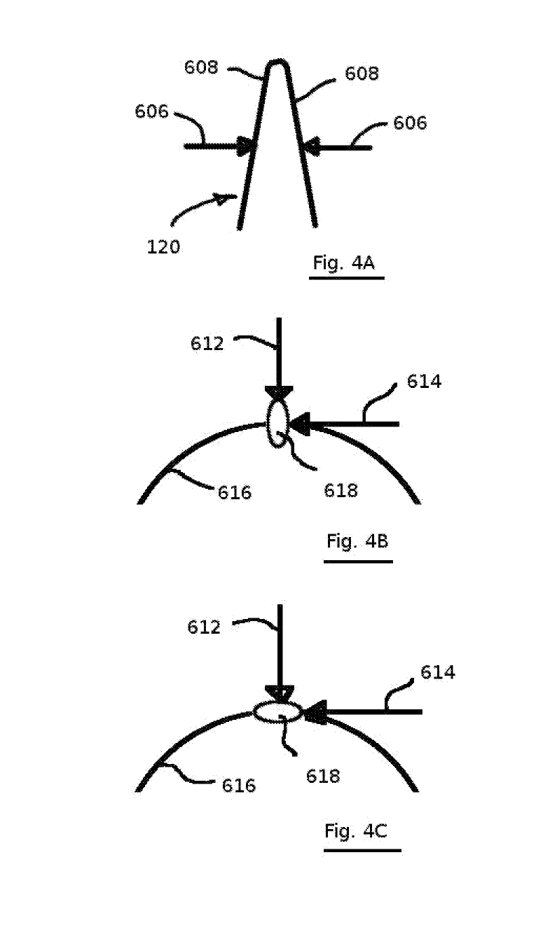

FIGS. 4A-4C are schematic views of loads applied to a stent having matched end stiffness in accordance with the present invention. FIG. 4A illustrates a pair of tangential applied loads; FIG. 4B illustrates a swaged stent cross section with the major axis perpendicular to the circumference of the stent; and FIG. 4C illustrates a swaged stent cross section with the minor axis perpendicular to the circumference of the stent.

Examples of applied loads include radial and tangential loads. Those skilled in the art will appreciate that radial applied loads and tangential applied loads can combine into a resultant load. In comparing the substantially equal stiffness of struts in response to an applied load, applied loads can be considered separately as either radial applied loads or tangential applied loads.

Referring to FIG. 4A, a pair of tangential applied loads 606 are applied to struts 608 of the stent 120. The pair of tangential applied loads 606 are tangential to the circumference of the stent and perpendicular to the stent axis. In one example, the pair of tangential applied loads 606 is generated by a vessel compressing the stent 120 towards a smaller circumference.

Referring to FIG. 4B, a swaged strut 618 with an ellipsoid cross section lies on the circumference 616 of a stent. The major axis of the ellipsoid is perpendicular to the circumference of the stent and the minor axis of the ellipsoid is tangential to the circumference of the stent. This orientation of the ellipsoid can be used to achieve matched end stiffness when the struts in the end portion of the stent are longer than the struts in the body portion and a radial applied load 612 is applied to the swaged strut 618. This orientation can also be used to achieve matched end stiffness when the struts in the end portion of the stent are shorter than the struts in the body portion and a tangential applied load 614 is applied to the swaged strut 618.

The radial applied load 612 is normal to the circumference of the stent and intersects the stent axis. The tangential applied load 614 is tangential to the circumference of the stent and perpendicular to the stent axis. In one example, the tangential applied load 614 is generated by a vessel compressing the stent towards a smaller circumference. A tangential applied load opposite the tangential applied load 614 illustrated can be generated when a balloon expands the stent.

Referring to FIG. 4C, a swaged strut 618 with an ellipsoid cross section lies on the circumference 616 of a stent. The major axis of the ellipsoid is tangential to the circumference of the stent and the minor axis of the ellipsoid is perpendicular to the circumference of the stent. This orientation of the ellipsoid can be used to achieve matched end stiffness when the struts in the end portion of the stent are longer than the struts in the body portion and a tangential applied load 614 is applied to the swaged strut 618. This orientation can also be used to achieve matched end stiffness when the struts in the end portion of the stent are shorter than the struts in the body portion and a radial applied load 612 is applied to the swaged strut 618.

Stiffness of the struts can be modeled as a simple beam. For a simple beam in bending, the stiffness (P/w) is:

$$\frac{P}{w} ∝ \frac{EI}{{L}^{3}}$$

where P is the applied load, w is the deflection, E is the modulus of elasticity of the strut material, I is the area moment of inertia of the strut cross section, and L is the strut length. This relation applies to a number of beams of uniform cross section, including many simply supported and cantilevered beams with center point or uniform loadings.

Struts can be determined to have substantially equal stiffnesses in a number of ways, by calculation or by experimentation. By calculation using the equation above, struts have substantially equal stiffnesses when the value of E₁I₁/(L₁)³ calculated for one strut (1) is within plus or minus five percent of the value of E₂I₂/(L₂)³ calculated for the another strut (2).

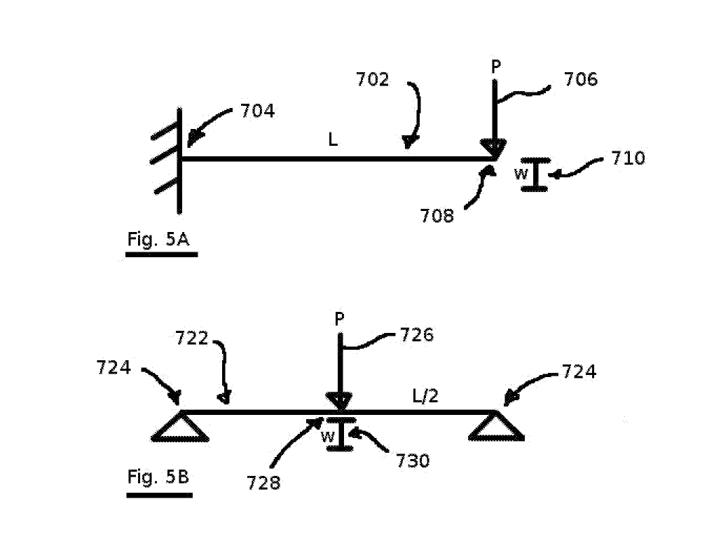

FIGS. 5A & 5B are schematic views of models of strut loading for a stent having matched end stiffness in accordance with the present invention. FIG. 5A illustrates an end loaded cantilevered beam and FIG. 5B illustrates a center loaded simply supported beam. Such beam models can be used to compare strut stiffnesses experimentally.

Referring to FIG. 5A, a cantilevered beam 702 is restrained at one end 704 and a load (P) 706 is applied at the unrestrained end 708 at the full length L of the cantilevered beam 702 and the deflection (w) 710 of the unrestrained end 708 is measured. The stiffness is determined by calculating P/w. Applying this model to compare strut stiffnesses experimentally, one strut of a stent is restrained at one end with the other end of the strut being unrestrained. A load P₁ is applied as at the unrestrained end of the strut at the full length L₁, a deflection w₁ of the unrestrained end of the strut is measured, and the stiffness is determined by calculating P₁/w₁. The procedure is repeated for another strut by applying a load P₂ at the unrestrained end of the strut at the full length L₂, measuring a deflection w₂ of the unrestrained end of the strut, and determining the stiffness by calculating P₂/w₂. The struts have substantially equal stiffnesses when the values P₁/w₁ and P₂/w₂ are within plus or minus five percent of each other.

Referring to FIG. 5B, a simply supported beam 722 is supported at ends 724 and a load (P) 726 is applied at the midpoint 728 at the half length L/2 of the cantilevered beam 722 and the deflection (w) 730 of the midpoint 728 is measured. The stiffness is determined by calculating P/w. Applying this model to compare strut stiffnesses experimentally, one strut of a stent is supported at both ends and a load P₁ applied at the midpoint, i.e., at L₁/2. The deflection w₁ from the load P₁ is measured at the midpoint. The procedure is repeated for another strut by applying a load P₂ at the midpoint of the strut at the half length L₂/2, measuring a deflection w₂ of the midpoint of the strut, and determining the stiffness by calculating P₂/w₂. The struts have substantially equal stiffnesses when the values P₁/w₁ and P₂/w₂ are within plus or minus five percent of each other.

FIGS. 6A-6D are detail cross section views of wire for a stent with matched end stiffness in accordance with the present invention. FIGS. 6A and 6B illustrate a solid wire, and FIGS. 6C and 6D illustrate a lumen wire.

Referring to FIG. 6A, the cross section of the wire 200 is round with a radius r. The round cross section of the wire 200 can be the initial cross section of the wire before stent fabrication and can be maintained in the body portion (body struts and/or body crowns) and/or the end crowns in the end portion. Referring to FIG. 6B, the cross section of the wire 210 is ellipsoid with a major axis radius a and a minor axis radius b. The ellipsoid cross section of the wire 210 can be the final cross section of the wire after swaging, such as the final cross section for the end portion (end struts and/or end crowns). In one embodiment, the cross section of the body struts is round, and the cross section of the end struts is ellipsoid with the major axis of the ellipsoid perpendicular to a circumference of the stent. In another embodiment, the cross section of the body struts is round, and the cross section of the end struts is ellipsoid with the minor axis of the ellipsoid perpendicular to a circumference of the stent. Those skilled in the art will appreciate that the cross section of the wire is not limited to round or ellipsoid and can be any cross section as desired for a particular application. For example, the wire can be square initially and rectangular after swaging.

The area moment of inertia can be selected to provide substantially equal stiffness in the struts regardless of strut length. The area moment of inertia of the round wire 200 is πr⁴/4 and the area moment of inertia for the ellipsoid wire 210 is πa³b/4. As described above for a simple beam in bending, the stiffness (P/w) is:

$$\frac{P}{w} ∝ \frac{EI}{{L}^{3}}$$

where P is the applied load, w is the deflection, E is the modulus of elasticity of the strut material, I is the area moment of inertia of the strut cross section, and L is the strut length. The stiffness (P/w) is proportional to the area moment of inertia I and inversely proportional to the cube of the strut length L. To achieve the same stiffness (P/w) when the length of a strut is doubled, the area moment of inertia must be increased by twice cubed, or eight times.

Assuming that the cross section of the wire remains constant during swaging, the initial cross sectional area of the circular wire (πr²) can be set equal to the final cross sectional area of the swaged ellipsoid wire (πab), and solved for the first equation r²=ab. Stiffness is proportional to I/L³ as noted above. To maintain equal stiffness between the circular wire of length L₁ and the ellipsoid wire of length L₂, I₁/(L₁)³ must equal I₂/(L₂)³, yielding the second equation I₁/I₂=(L₁/L₂)³. The moment of inertia I₁ for the circular wire is given by the third equation πr⁴/4 and the moment of inertia I₂ for the ellipsoid wire is given by the fourth equation πa³b/4 when the load is applied along the major axis a and the neutral bending axis is along the minor axis b. Combining the first through fourth equations: a²=r²(L₂/L₁)³ and b=r²/a. These equations can be used to calculate the dimensions of the swaged ellipsoid wire with changing length. Each example assumes an initial radius of 10 units for the circular wire. When the ratio of the lengths (L₂/L₁) is 1.20, the major axis a is 13.1 units and the minor axis b is 7.6 units to maintain equal stiffness between the circular wire strut and the longer ellipsoid strut. When the ratio of the lengths (L₂/L₁) is 1.50, the major axis a is 18.4 units and the minor axis b is 5.4 units to maintain equal stiffness. When the ratio of the lengths (L₂/L₁) is 1.80, the major axis a is 24.1 units and the minor axis b is 4.1 units to maintain equal stiffness. Those skilled in the art will appreciate that a similar calculation can be performed when the circular wire strut is longer than the ellipsoid strut.

The major and minor axes of the swaged ellipsoid strut can be perpendicular or tangential to the circumference of the stent depending on the relative length of the circular wire strut and the swaged ellipsoid strut, and the direction of loading. In application, the load can be applied radially or tangentially to a strut, and the swaged ellipsoid strut in the end portion of the stent can be longer, shorter, or equal in length to the circular wire strut in the body portion of the stent.

For a tangential applied load when the swaged ellipsoid strut is longer than the circular wire strut, the major axis of the swaged ellipsoid strut is tangential to the circumference of the stent. For a tangential applied load when the swaged ellipsoid strut is shorter than the circular wire strut, the major axis of the swaged ellipsoid strut is perpendicular to the circumference of the stent. For a radial applied load when the swaged ellipsoid strut is longer than the circular wire strut, the major axis of the swaged ellipsoid strut is perpendicular to the circumference of the stent. For a radial applied load when the swaged ellipsoid strut is shorter than the circular wire strut, the major axis of the swaged ellipsoid strut is tangential to the circumference of the stent.

FIGS. 6C and 6D illustrate a round and ellipsoid lumen wire, respectively. The round lumen wire 220 can be the initial cross section of the wire before stent fabrication and the ellipsoid lumen wire 230 can be the final cross section of the wire and portions of the stent, such as the end struts, after swaging. The calculation of the area moment of inertia I for these cross sections must account for the absence of structural matter within the lumen 224. Referring to FIG. 6C, the round drug-filled wire 220 has a wall 222 which defines a lumen 224 within the round lumen wire 220. Referring to FIG. 6D, the wall 222 and the lumen 224 of the ellipsoid lumen wire 230 have changed to an ellipsoid shape from the swaging of the round lumen wire 220. The lumen 224 can be left empty or can include a drug or other therapeutic agent to treat the patient in which the stent is implanted. The wall 222 can include holes or perforations (not shown) to allow the drug to exit the drug-filled lumen 224.

The drug can be any biologically or pharmacologically active substance, and may include, but is not limited to, antineoplastic, antimitotic, anti-inflammatory, antiplatelet, anticoagulant, antifibrin, antithrombin, antiproliferative, antibiotic, antioxidant, and antiallergic substances as well as combinations thereof. Examples of such antineoplastics and/or antimitotics include paclitaxel (e.g., TAXOL® by Bristol-Myers Squibb Co., Stamford, Conn.), docetaxel (e.g., Taxotere® from Aventis S. A., Frankfurt, Germany), methotrexate, azathioprine, vincristine, vinblastine, fluorouracil, doxorubicin hydrochloride (e.g., Adriamycin® from Pharmacia & Upjohn, Peapack N.J.), and mitomycin (e.g., Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.). Examples of such cytostatic or antiproliferative agents include ABT-578 (a synthetic analog of rapamycin), rapamycin (sirolimus), zotarolimus, everolimus, angiopeptin, angiotensin converting enzyme inhibitors such as captopril (e.g., Capoten® and Capozide® from Bristol-Myers Squibb Co., Stamford, Conn.), cilazapril or lisinopril (e.g., Prinivil® and Prinzide® from Merck & Co., Inc., Whitehouse Station, N.J.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other biologically or pharmacologically active substances or agents that may be used include nitric oxide, alpha-interferon, genetically engineered epithelial cells, and dexamethasone. In other examples, the biologically or pharmacologically active substance is a radioactive isotope for implantable device usage in radiotherapeutic procedures. Examples of radioactive isotopes include, but are not limited to, phosphorus (P³²), palladium (Pd¹⁰³), cesium (Cs¹³¹), Iridium (I¹⁹²) and iodine (I¹²⁵). While the preventative and treatment properties of the foregoing biologically or pharmacologically active substances are well-known to those of ordinary skill in the art, the biologically or pharmacologically active substances are provided by way of example and are not meant to be limiting.

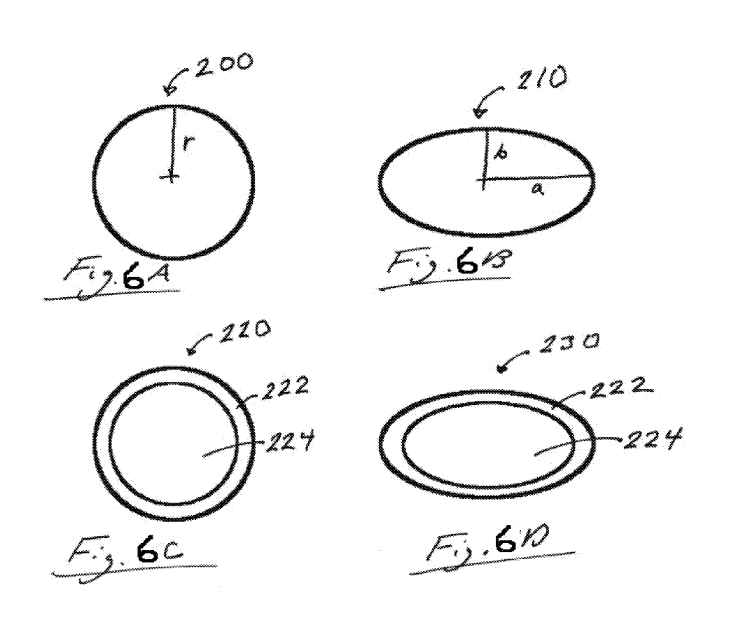

FIGS. 7A-7C are detail views of swaged wire for a stent with matched end stiffness in accordance with the present invention. In these examples, the end portion has been swaged to make the shorter end struts broader and thinner, thus maintaining substantially equal stiffness over the end portion in response to a radial applied load. The exemplary end portion is illustrated with three portions which will be fabricated into six end struts alternating with three peak end crowns and three valley end crowns as illustrated in FIG. 2C.

Referring to FIG. 7A, the wire 300 includes a body portion 302, a transition crown portion 304, and an end portion 310. The area moment of inertia of the end portion 310 increases stepwise in a direction away from the body portion 302. The end portion 310 includes a first step 312 which will be fabricated into the longest end struts with a peak and a valley end crown, a second step 314 which will be fabricated into the medium end struts with a peak and a valley end crown, and a third step 316 which will be fabricated into the shortest end struts with a peak and a valley end crown.

Referring to FIG. 7B, the wire 320 includes a body portion 322, a transition crown portion 324, and an end portion 330. The area moment of inertia of the end portion 330 increases continuously in a direction away from the body portion 322. The end portion 330 includes a first portion 332 which will be fabricated into the longest end struts with a peak and valley end crown, a second portion 334 which will be fabricated into the medium end struts with a peak and valley end crown, and a third portion 336 which will be fabricated into the shortest end struts with a peak and valley end crown.

Referring to FIG. 7C, the wire 340 includes a body portion 342, a transition crown portion 344, and an end portion 350. The end portion 350 includes swaged end struts 352 and unswaged end crowns 354, which increases the visibility of the end crowns under fluoroscopy. In one embodiment, the unswaged end crowns maintain the same cross section as the struts in the body portion 342. In this example, the length of each pair of swaged end struts 352 decreases in a direction away from the body portion 342, so the area moment of inertia of each pair of swaged end struts 352 decreases in the direction away from the body portion 342 to maintain substantially equal stiffness (P/w). In one embodiment, the cross section of the body portion 342 and transition crown portion 344 is round, the cross section of the end struts 352 is ellipsoid with the minor axis of the ellipsoid perpendicular to a circumference of the stent, and the cross section of the end crowns 354 is round.

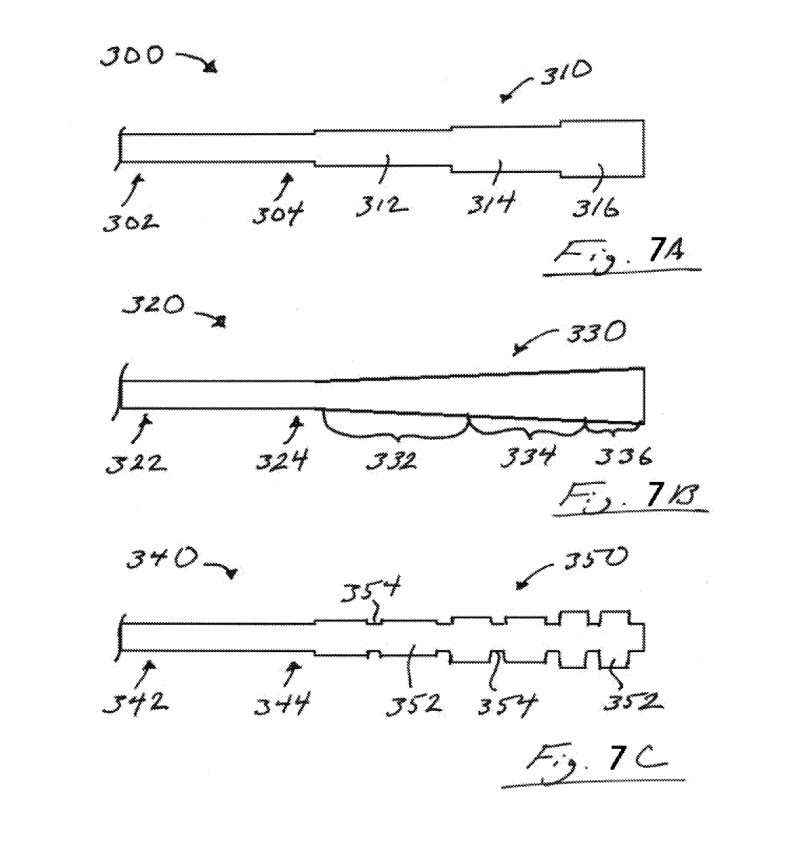

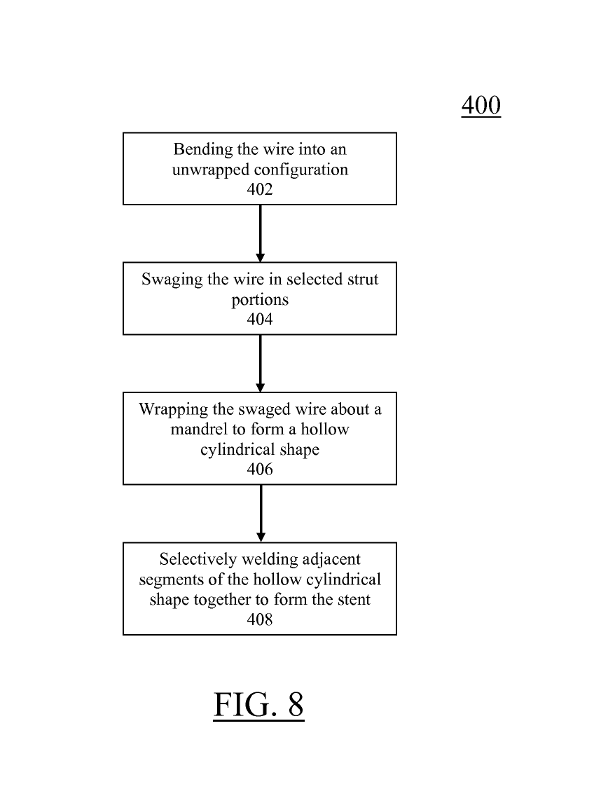

FIG. 8 is a flowchart of a method of manufacturing a stent with matched end stiffness in accordance with the present invention. The method 400 is a method of manufacturing a stent from a wire, the stent having a body portion and an end portion. The method 400 includes bending the wire into an unwrapped configuration 402; swaging the wire in selected strut portions 404 in the end portion of the wire, the degree of swaging being selected so that each end strut in the end portion of the stent has a stiffness in response to an applied load substantially equal to a stiffness in response to the applied load of body struts in the body portion of the stent; wrapping the swaged wire about a mandrel to form a hollow cylindrical shape 406; and selectively welding adjacent segments of the hollow cylindrical shape together to form the stent 408. Those skilled in the art will appreciate that the steps of the method 400 can be performed in different orders as desired for a particular application. In one example, the bending 402 is performed before the swaging 404. In another example, the swaging 404 is performed before the bending 402.

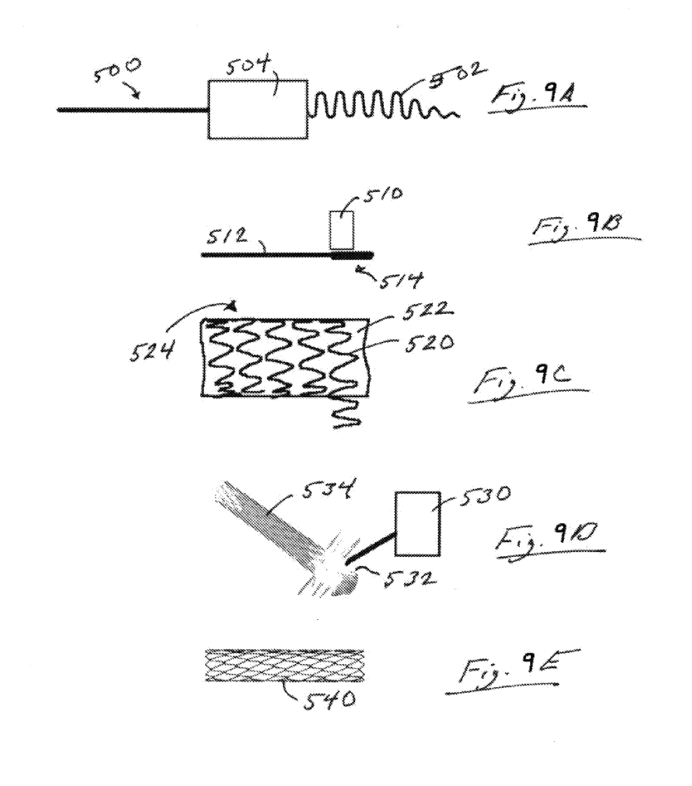

FIGS. 9A-9E are schematic views of manufacture of a stent with matched end stiffness in accordance with the present invention.

Referring to FIG. 9A, a bending device 504 bends a wire 500 into an unwrapped configuration 502. Referring to FIG. 9B, the wire in the unwrapped configuration 512 swaged in selected strut portions in the end portion 514 of the wire with a press 510, such as a hammer-type press, roller mill, a die, or the like, to achieve the desired cross section and area moment of inertia for the selected areas. The swaging produces substantially equal stiffness for all struts in the stent by applying a selected degree of swaging, so that each end strut in the end portion 514 of the stent has a stiffness substantially equal to stiffness of body struts in the body portion of the stent. In one example, the wire is round and the swaged areas are ellipsoid. In FIG. 9C, the swaged wire 520 is wrapped about a mandrel 522 to form a hollow cylindrical shape 524. In FIG. 9D, a welder 530 selectively welds adjacent segments of the hollow cylindrical shape 532 together to form the stent 534. FIG. 9E illustrates the completed stent 540 with matched end stiffness.

It is important to note that FIGS. 1-9 illustrate specific applications and embodiments of the present invention, and are not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system reacting to an applied load, the stent delivery system comprising:
a catheter;
a balloon operably attached to the catheter; and
a stent disposed on the balloon;
wherein the stent comprises:
a wire bent into a waveform having a constant frequency and wrapped into a hollow cylindrical shape to form the stent, the wire comprising:
a body portion having body struts connected between body crowns, the body struts having substantially equal lengths, and the waveform in the body portion having a constant amplitude; and
at least one end portion attached to the body portion, the at least one end portion having end struts connected between end crowns, the waveform in the at least one end portion having an amplitude different from the constant amplitude of the waveform in the body portion;
wherein the cross sections of the end struts are selected so that the body struts and the end struts have substantially equal stiffnesses in response to the applied load.

2. The stent delivery system of claim 1 wherein the waveform is sinusoidal.

3. The stent delivery system of claim 1 wherein the cross section of the body struts is round, and the cross section of the end struts is an ellipsoid with the major axis of the ellipsoid perpendicular to a circumference of the stent.

4. The stent delivery system of claim 3 wherein the end struts are longer than the body struts and the applied load is a radial applied load.

5. The stent delivery system of claim 3 wherein the end struts are shorter than the body struts and the applied load is a tangential applied load.

6. The stent delivery system of claim 1 wherein the cross section of the body struts is round, the cross section of the end struts is an ellipsoid with the minor axis of the ellipsoid perpendicular to a circumference of the stent.

7. The stent delivery system of claim 6 wherein the end struts are longer than the body struts and the applied load is a tangential applied load.

8. The stent delivery system of claim 6 wherein the end struts are shorter than the body struts and the applied load is a radial applied load.

9. The stent delivery system of claim 1 wherein the wire has a wall defining a lumen within the wire.

10. The stent delivery system of claim 9 wherein the lumen is a drug-filled lumen.

11. The stent delivery system of claim 1 wherein at least one of the body crowns is welded to a body crown in an adjoining segment of the hollow cylindrical shape.

12. A stent comprising:
a wire bent into a waveform having a constant frequency and wrapped into a hollow cylindrical shape to form the stent, the wire comprising:
a body portion having body struts connected between body crowns, the body struts having substantially equal lengths, and the waveform in the body portion having a constant amplitude; and
at least one end portion attached to the body portion, the at least one end portion having end struts connected between end crowns, the waveform and the at least one end portion having an amplitude different from the constant amplitude of the waveform in the body portion;
wherein the cross sections of the end struts are selected so that the body struts and the end struts have substantially equal stiffnesses in response to the applied load.

13. The stent of claim 12 wherein the waveform is sinusoidal.

14. The stent of claim 12 wherein the cross section of the body struts is round, and the cross section of the end struts is an ellipsoid with the major axis of the ellipsoid perpendicular to a circumference of the stent.

15. The stent delivery system of claim 14 wherein the end struts are longer than the body struts and the applied load is a radial applied load.

16. The stent delivery system of claim 14 wherein the end struts are shorter than the body struts and the applied load is a tangential applied load.

17. The stent of claim 12 wherein the cross section of the body struts is round, the cross section of the end struts is an ellipsoid with the minor axis of the ellipsoid perpendicular to a circumference of the stent.

18. The stent delivery system of claim 17 wherein the end struts are longer than the body struts and the applied load is a tangential applied load.

19. The stent delivery system of claim 17 wherein the end struts are shorter than the body struts and the applied load is a radial applied load.

20. The stent of claim 12 wherein the wire has a wall defining a lumen within the wire.

21. The stent of claim 20 wherein the lumen is a drug-filled lumen.

22. The stent of claim 12 wherein at least one of the body crowns is welded to a body crown in an adjoining segment of the hollow cylindrical shape.

23. A method of manufacturing a stent from a wire, the stent having a body portion and an end portion, the method comprising:
bending the wire into an unwrapped configuration;
swaging the wire in selected strut portions in the end portion of the wire, the degree of swaging being selected so that each end strut in the end portion of the stent has a stiffness in response to an applied load substantially equal to a stiffness in response to the applied load of body struts in the body portion of the stent;
wrapping the swaged wire about a mandrel to form a hollow cylindrical shape; and
selectively welding adjacent segments of the hollow cylindrical shape together to form the stent.

24. The method of claim 23 wherein the bending is performed before the swaging.
